(19) United States
(12) Patent Application Publication
(10) Pub. No.: US 2025/0340941 A1
(43) Pub. Date: Nov. 6, 2025

(54) ASSAY AND METHODS FOR DRUG DISCOVERY

(71) Applicant: BIOVIE INC., Carson City, NV (US)

(72) Inventors: Clarence Nathaniel Ahlem, San Diego, CA (US); Christopher L. Reading, San Diego, CA (US)

(21) Appl. No.: 19/265,505

(22) Filed: Jul. 10, 2025

Related U.S. Application Data
(63) Continuation of application No. PCT/US2024/011212, filed on Jan. 11, 2024.
(60) Provisional application No. 63/479,973, filed on Jan. 13, 2023.

(51) Int. Cl. C12Q 1/6883; G01N 33/50; G01N 33/68

(52) U.S. Cl. CPC C12Q 1/6883; G01N 33/5023; G01N 33/5026; G01N 33/5041; G01N 33/6863; C12Q 2600/136; C12Q 2600/154; G01N 2333/4703; G01N 2333/7151; G01N 2500/10

(57) ABSTRACT

Disclosed herein are methods for assaying a potential drug candidate for the treatment, prevention, reduction or amelioration of neurodegenerative diseases and disorders. Some aspects pertain to stimulating a cell to induce a phenotype characteristic of a neurodegenerative disease or disorder and contacting the cell with a potential drug candidate and determining a responsive change, wherein a decrease or loss in the phenotype is indicative that the drug candidate is capable of treating, preventing, reducing or ameliorating neurodegenerative diseases or disorders.

### INCORPORATION BY REFERENCE TO ANY PRIORITY APPLICATIONS

This application is a continuation of PCT Application No. PCT/US2024/011212, filed Jan. 11, 2024, and claims the benefit of priority of U.S. Provisional Application No. 63/479,973, filed Jan. 13, 2023. The foregoing application is incorporated herein by reference in its entirety for all purposes.

### FIELD OF THE DISCLOSURE

This disclosure relates to assays and methods for drug discovery. More specifically, an in vitro screening method to identify a potential drug candidate capable of treating, preventing, reducing, or ameliorating a neurodegenerative disorder or disease.

### BACKGROUND

Neurodegenerative diseases, such as Parkinson's disease (PD), are one of the most serious global health concerns. Global estimates show over 8.5 million individuals with PD, and the prevalence of PD has doubled over the past 25 years. More broadly, neurodegenerative diseases affect approximately 50 million Americans annually. A core research challenge is identifying and developing drugs that can treat, prevent, reduce, or ameliorate the loss of neural cells and nervous system dysfunction.

Animal models can provide useful means of assessing the efficacy of potential drug treatments. However, using animal models has several drawbacks, such that animal models are not amenable to high throughput screening: (1) the cost of purchasing, raising, and maintaining the large number of animals required during the course of the experiment; (2) the technical difficulty of inducing disease types, such as PD, in rats is high and carries a significant risk of failure or death of the experimental subject; and (3) the dose required for candidate drugs is often high, which disfavors screening of drug candidates that are costly or difficult to synthesize or acquire.

Thus, there is a need for cell-based in vitro assay methods that can be easily performed and give reliable results. The solution to the technical problem described is achieved by providing the embodiments characterized in the claims and described further below.

### SUMMARY OF THE DISCLOSURE

Some embodiments of the disclosure relate to an in vitro screening method to identify a potential drug candidate capable of treating, preventing, reducing, or ameliorating a disorder or disease. In some embodiments, the method includes providing a sample for stimulation selected from the group consisting of a cell, tissue, blood, monocytes, microglia, macrophages, adipocytes, neuroblastoma, pheochromocytoma, and Lund human mesencephalic (LUHMES) cells, stimulating the sample with an agonist to induce a phenotype or phenotypic reaction, wherein the phenotype or phenotypic reaction substantially corresponds to a disease or condition associated with a biological clock, contacting the one or more cells exhibiting the phenotype or phenotypic reaction with one or more potential drug candidates, determining a responsive change in the phenotype of the sample, and providing the drug candidate to a subject to treat, reduce, prevent, or ameliorate a disease or condition associated with a biological clock in a subject. In some embodiments, the responsive change is a decrease or loss in the phenotype and the decrease or loss is indicative that the potential drug candidate is capable of preventing, reducing, or ameliorating a neurodegenerative disorder or disease. In some embodiments, the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism. In some embodiments, the disease or condition associated with biological clocks in the subject in need thereof is based on modulation of DNA methylation of genes associated with biological clocks. In some embodiments, the disease or condition associated with a biological clock in the subject in need thereof is associated with genes or genomic regions hypermethylated with age. In some embodiments, the disease or condition associated with a biological clock in the subject in need thereof is associated with genes or genomic regions hypomethylated with age. In some embodiments, the disease or condition associated with inflammatory TNF signaling. In some embodiments, the disease or condition associated with inflammatory NF-kB signaling. In some embodiments, the disease or condition associated with a biological clock in a subject in need thereof is associated with Tau phosphorylation. In some embodiments, the disease or condition associated with a biological clock in a subject in need thereof is associated with hyperglycemia. In some embodiments, the disease or condition associated with a biological clock in a subject in need thereof is associated with hyperinsulinemia. In some embodiments, the disease or condition associated with a biological clock in a subject in need thereof is associated with obesity. In some embodiments, the disease or condition associated with a biological clock in a subject is connected to leptin. In some embodiments, the subject experiences a decrease between about a 5% to about a 100% reduction in conditions or symptoms connected to leptin after administration of the drug candidate and at least one pharmaceutically acceptable excipient. In some embodiments, the disease or condition associated with a biological clock is associated with a subject's SkinBloodAge. In some embodiments, the subject experiences a decrease between about a 5% to about a 100% reduction in conditions or symptoms connected to SkinBloodAge after administration of the drug candidate and at least one pharmaceutically acceptable excipient. In some embodiments, the disease or condition associated with a biological clock is associated with a subject's dinucleotide (CpG) methylation in association with leptin promotor (DNAmLeptin). In some embodiments, the subject experiences a decrease between about a 5% to about a 100% reduction in conditions or symptoms connected to DNAmLeptin after administration of the drug candidate and at least one pharmaceutically acceptable excipient. In some embodiments, the disease or condition associated with a biological clock is associated with a subject's DNAmPackYears. In some embodiments, the subject experiences a decrease between about a 5% to about a 100% reduction in conditions or symptoms connected to DNAmPackYears after administration of the drug candidate and at least one pharmaceutically acceptable excipient.

Some embodiments relate to a method to treat, prevent, reduce, or ameliorate a neurodegenerative disorder or disease. In some embodiments, the method includes providing a sample for stimulation selected from the group consisting of a cell, tissue, blood, monocytes, microglia, macrophages, adipocytes, neuroblastoma, pheochromocytoma, and Lund human mesencephalic (LUHMES) cells, stimulating the sample with an agonist to induce a phenotype or phenotypic reaction, wherein the phenotype or phenotypic reaction substantially corresponds to a disease or condition associated with a biological clock, contacting the one or more cells exhibiting the phenotype or phenotypic reaction with one or more potential drug candidates, determining a responsive change in the phenotype of the sample, and providing the drug candidate to a subject to treat, reduce, prevent, or ameliorate a disease or condition associated with a biological clock in a subject. In some embodiments, the responsive change is a decrease or loss in the phenotype and the decrease or loss is indicative that the potential drug candidate is capable of preventing, reducing, or ameliorating a neurodegenerative disorder or disease. In some embodiments, the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism. In some embodiments, the disease or condition associated with biological clocks in the subject in need thereof is based on modulation of DNA methylation of genes associated with biological clocks. In some embodiments, the disease or condition associated with a biological clock in the subject in need thereof is associated with genes or genomic regions hypermethylated with age. In some embodiments, the disease or condition associated with a biological clock in the subject in need thereof is associated with genes or genomic regions hypomethylated with age. In some embodiments, the disease or condition associated with inflammatory TNF signaling. In some embodiments, the disease or condition associated with inflammatory NF-kB signaling. In some embodiments, the disease or condition associated with a biological clock in a subject in need thereof is associated with Tau phosphorylation. In some embodiments, the disease or condition associated with a biological clock in a subject in need thereof is associated with hyperglycemia. In some embodiments, the disease or condition associated with a biological clock in a subject in need thereof is associated with hyperinsulinemia. In some embodiments, the disease or condition associated with a biological clock in a subject in need thereof is associated with obesity. In some embodiments, the disease or condition associated with a biological clock in a subject is connected to leptin. In some embodiments, the subject experiences a decrease between about a 5% to about a 100% reduction in conditions or symptoms connected to leptin after administration of the drug candidate and at least one pharmaceutically acceptable excipient. In some embodiments, the disease or condition associated with a biological clock is associated with a subject's SkinBloodAge. In some embodiments, the subject experiences a decrease between about a 5% to about a 100% reduction in conditions or symptoms connected to SkinBloodAge after administration of the drug candidate and at least one pharmaceutically acceptable excipient. In some embodiments, the disease or condition associated with a biological clock is associated with a subject's dinucleotide (CpG) methylation in association with leptin promotor (DNAmLeptin). In some embodiments, the subject experiences a decrease between about a 5% to about a 100% reduction in conditions or symptoms connected to DNAmLeptin after administration of the drug candidate and at least one pharmaceutically acceptable excipient. In some embodiments, the disease or condition associated with a biological clock is associated with a subject's DNAmPack Years. In some embodiments, the subject experiences a decrease between about a 5% to about a 100% reduction in conditions or symptoms connected to DNAmPack Years after administration of the drug candidate and at least one pharmaceutically acceptable excipient.

### BRIEF DESCRIPTION OF THE DRAWINGS

FIG. 1 shows the effect of NE3107 on TNFa-stimulated TNFR1 ERK-dependent phosphorylation. THP-1 monocytes were treated with 100 nM NE3107, 1 uM IKK-16 (IKK inhibitor), 1 uM Tpl2 Kinase inhibitor (TPL2i) or Vehicle (0.1% DMSO) and stimulated with TNFa 50 ng/mL. Cell lysates were collected at 0, 2, 5, 10, 15, and 30 minutes. The results are given as fold change with respect to the control following Western blotting and quantification of band intensities by densitometry scanning.

FIG. 2 illustrates a chart representing a Phase 3, randomized, placebo-controlled trial of NE3107 (17α-ethynylandrost-5-ene-3ϕ3,7ϕ3,17ϕ3-triol) in subjects with mild to moderate probable Alzheimer's disease.

FIGS. 3A-3G illustrate graphs for improvements in the blinded assessments from the Phase 3, Randomized, Placebo-Controlled Trials.

FIGS. 4A-4B illustrates graphs for imaging sub-studies of vMRI hippocampus volume and amygdala volume in the blinded assessments from the Phase 3, Randomized, Placebo-Controlled Trials.

FIG. 5 illustrates graphs for imaging sub-studies FDG-PET in the blinded assessments from the Phase 3, Randomized, Placebo-Controlled Trials.

FIGS. 6A-6B illustrates graphs for neuropsychiatric inventory in the blinded assessments from the Phase 3, Randomized, Placebo-Controlled Trials.

### DETAILED DESCRIPTION OF THE DISCLOSURE

The present disclosure can be understood more readily by referencing the following detailed description, examples, drawings, and claims, and their previous and following descriptions. However, before the present methods are disclosed and described, it is to be understood that this disclosure is not limited to the specific methods disclosed unless otherwise specified. No single component or collection of components is essential or indispensable. For example, in some embodiments one or more variables, such as Y or Y and Q may be omitted. It is also to be understood that the terminology used herein is for the purpose of describing particular aspects only and is not necessarily intended to be limiting.

This description is provided as an enabling teaching of the disclosure. To this end, those skilled in the relevant art will recognize and appreciate that many changes can be made to the various aspects of the disclosure described herein while still obtaining beneficial results. It will also be apparent that some of the desired benefits can be obtained by selecting some of the features described herein without utilizing other features. Accordingly, those who work in the art will recognize that many modifications and adaptations to the present description are possible and can even be desirable in certain circumstances and are a part of the present disclosure. Thus, this description is provided as illustrative of certain principles of the present disclosure and not in limitation thereof.

### Definitions

Unless defined otherwise, all technical and scientific terms used herein have the same meaning as is commonly understood by one of ordinary skill in the art. All patents, applications, published applications, and other publications referenced herein are incorporated by reference in their entirety unless stated otherwise. In the event that there are a plurality of definitions for a term herein, those in this section prevail unless stated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the term “agonist” is a substance that interacts with a cellular constituent to produce a biological response. The response may be the induction of a cellular phenotype. An agonist could be any molecule that mimics a biological activity of an endogenous molecule, such as a chemokine.

As used herein, the term “gene expression” refers to gene information encoded by a gene via “transcription” of the gene (i.e., via the enzymatic action of RNA polymerase), such as RNA (e.g., mRNA, rRNA, (RNA, or snRNA), and in the case of a gene encoding a protein, it means a process of converting to a protein via mRNA “translation.” At many stages in this process gene expression can be regulated. As used here, “activation” means regulation that increases the production of a gene expression product (i.e., RNA or protein).

As used herein, the term “ERK” refers to extracellular signal regulated kinase. ERK refers to all isoforms of the ERK enzyme including, but not limited to, ERK 1, ERK2, ERK3, ERK4, ERK5, ERK6 and ERK7 and includes all variants, isoforms and species homologs thereof.

As used herein, the term “NF-kB” means nuclear factor kappa B and refers to any of the family of transcription factor complexes which have as at least one of the components the subunits known as p65 (ReIA), p50, p52, c-rel, p68 (ReiB) and includes all variants, isoforms and species homologs thereof.

As used herein, “activation of NF-kB” means activation of genes associated with the inflammatory state resulting from binding of an NF-kB transcription factor complex to DNA elements, including, but not limited to, the kB element in the immunoglobulin kappa light chain gene. Interaction with its endogenous inhibitor IkB typically retains the NF-kB complex in the cytoplasm. Activation of NF-kB must be preceded by localization of the NF-kB complex to the nucleus. Translocation of the NF-kB complex to the nucleus does not constitute NF-kB activity unless transcription from genes associated with the inflammatory state is stimulated.

As used herein, the term “p38 MAPK” refers to p38 mitogen-activated protein kinases and includes all variants, isoforms and species homologs thereof. P38 MAPKs are members of the MAPK family that are activated by a variety of environmental stresses and inflammatory cytokines.

As used herein, the term “MCP-1” is monocyte chemoattractant protein lor chemokine (CC-motif) ligand 2 (CCL2) and includes all variants, isoforms, and species homologs thereof.

As used herein, the term “IKK” refers to I kappa B kinase, an enzyme complex that is involved in propagating the cellular response to inflammation and is part of the upstream NF-kB signal transduction cascade.

As used herein, the term “Tpl2” refers to tumor progression locus 2, a serine-threonine kinase that regulates inflammatory pathways.

As used herein, the term “drug candidate” is any substance that is evaluated for its ability to treat, prevent, reduce, or ameliorate a neurodegenerative disease or disorder in a subject. In some embodiments, the drug candidate may be a “drug” as the term is defined in the Food and Drug Cosmetic Act, § 321(g)(1). Drug candidates include, but are not limited to, chemical compounds, biological agents, proteins, peptides, nucleic acids, lipids, polysaccharides and immunomodulators.

As used herein, “a decrease or loss in a phenotype” refers to altering a phenotype such that is more closely approximates a normal phenotype, which refers to a phenotype that falls within a range of phenotypes found in healthy cells that are not affected by a neurodegenerative disease or disorder.

As used herein, “contacting” or “stimulating” or like terms refers to bringing into proximity such that a molecular interaction can take place. For example, contacting refers to bringing at least two compositions, molecules, substances, cells, articles, or things into contact, i.e., such that they are in proximity to mix or touch.

As used herein, “assaying” or “assay” or like terms refer to the determination of a characteristic of a substance, such as a molecule, composition, compound, or cell, upon stimulation with one or more external stimuli.

As used herein, “high-throughput screening” refers to rapid in vitro assays of large numbers of samples.

The terms “treatment,” “treating,” “treat,” and the like shall be given their ordinary meaning and shall also include herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. The terms “treatment,” as used herein shall be given its ordinary meaning and shall also cover any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; and/or (c) relieving the disease symptom, e.g., causing regression of the disease or symptom.

All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the general description and the following detailed description are exemplary and explanatory only and are not restrictive. The term “and/or” denotes that the provided possibilities can be used together or be used in the alternative. Thus, the term “and/or” denotes that both options exist for that set of possibilities.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the disclosure. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Methods and Uses

Aspects of the present disclosure relate to an in vitro screening method to identify a potential drug candidate. In some embodiments, the in vitro screening method may identify a potential drug candidate capable of treating, preventing, reducing, or ameliorating a disorder or disease. In some embodiments, the disorder or disease is a neurodegenerative disorder or disease. In some embodiments, the neurodegenerative disease or condition is dementia.

In some embodiments, the in vitro screening method may include providing a sample for stimulation. In some embodiments, the sample is a cell. In some embodiments, the sample is tissue. In some embodiments, the sample is blood. In some embodiments, the sample includes monocytes. In some embodiments, the sample includes microglia. In some embodiments, the monocytes include, but are not limited to, CX3CR1^{low}, CCR2^{pos}, Ly6C^{high}, PD-L1^{neg}, CD14⁺⁺, CD16⁺, CD14^{dim}, CD16⁺, CD16⁻ CX3CR1^{high}, CCR2^{neg}, Ly6C^{low}, PD-L1^{pos}. In some embodiments, the cells are T cells or granulocytes. In some embodiments, the cells are NK cells or granulocytes. In some embodiments, the cell for stimulation may be selected from the group consisting of, but not limited to, THP-1 human monocytes, RAW 264.7 macrophages, 3T3-L1 adipocytes, SH-SY5Y neuroblastoma, PC-12 pheochromocytoma and Lund human mesencephalic (LUHMES) cells. In some embodiments, the cell for stimulation is a different neural cell line. In some embodiments, the cell for stimulation is a glial cell line. In some embodiments, the cell for stimulation is an immune cell. In some embodiments, the immune cell may be selected from, but is not limited to, dendritic cells, B cells, or T-cell subsets. In some embodiments, the cell for stimulation is a stem cell. In some embodiments the stem cell is an embryonic or induced pluripotent stem cell. In some embodiments, the cell for stimulation is an organ-specific cell. In some embodiments, the organ-specific cell may include, but is not limited to, hepatocytes or cardiomyocytes.

In some embodiments, the in vitro screening method may include stimulating the cell with an agonist to induce a phenotype or a phenotypic change. In some embodiments, the agonist for stimulating the cell may include, but is not limited to, tumor necrosis factor alpha or lipopolysaccharide. In some embodiments, the phenotype or phenotypic change is associated with a subject's SkinBloodAge clock. In some embodiments, the phenotype or phenotypic change is associated with a subject's dinucleotide (CpG) methylation associated with leptin promotor (DNAmLeptin). In some embodiments, the phenotype or phenotypic change is associated with a subject's DNAmPackYears. In some embodiments, the phenotype may correspond to a phenotype of a cell or tissue affected by a neurodegenerative disease or disorder. In some embodiments, the phenotypic change may be associated with biomarkers associated in gene expression profiles. In some embodiments, biomarkers associated in gene expression profiles include protein activity or cellular metabolism. In some embodiments, the phenotypic change may include phenotypes or changes related to one or more biological aging health clocks. In some embodiments, the phenotypic change may be associated with changes induced by environmental stressors or conditions mimicking physiological stress relevant to disease pathology.

In some embodiments, the neurodegenerative disorder or disease may be selected from the group consisting of, but not limited to, Alzheimer's disease, Parkinson's disease, amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration, Huntington's Disease, Spinocerebellar Ataxias, Lewy Body Dementia, Prion Diseases, Spinal Muscular Atrophy, Friedreich's Ataxia, Batten Disease, and vascular parkinsonism.

In some embodiments, the in vitro screening method may include contacting the one or more cells or tissue exhibiting the phenotype with the potential drug candidate. In some embodiments, the in vitro screening method may include contacting the one or more cells exhibiting the phenotype with the potential drug candidate in parallel in a high-throughput screening method. In other embodiments, the in vitro screening method may include contacting the one or more cells exhibiting the phenotype with one or more potential drug candidates in parallel in a high throughput screening method. In still other embodiments, the in vitro screening method may include contacting the one or more cells exhibiting the phenotype with the potential drug candidate sequentially. In some embodiments, the in vitro screening method may include varying durations and concentrations of drug exposure to assess dose-response relationships. In some embodiments, the in vitro screening method may include testing the effects of combining multiple drug candidates on the cells or tissues. In some embodiments, the in vitro screening method may include evaluating the long-term impact of drug exposure on cell phenotype, including potential delayed effects. In some embodiments, the in vitro screening method may include studying the ability of cells or tissues to revert to their original state after drug withdrawal. In some embodiments, the in vitro screening method may include assessing a range of phenotypic changes beyond gene expression and specific protein activations, such as alterations in cell morphology, viability, metabolic activity, or signal transduction pathways. In some embodiments, the in vitro screening method may include comparing the effects of potential drug candidates with known drugs or treatments to gauge efficacy or side effects. In some embodiments, the in vitro screening method may include including environmental factors (for example, oxidative stress or hypoxic conditions) in the assay to mimic disease conditions more closely. In some embodiments, the in vitro screening method may include utilizing advancing imaging techniques and automates image analysis to assess phenotypic changes at a cellular or sub-cellular level. In some embodiments, the in vitro screening method may include testing the effects of drug candidates on cells from different species to assess cross-species efficacy and safety. In some embodiments, the in vitro screening method may include integrating data on the activation or inhibition of specific molecular pathways to better understand the drug's mechanism of action.

In some embodiments, the in vitro screening method may include determining a responsive change in the cell phenotype. In some embodiments, the responsive change may be a decrease, reduction, or loss in the cell phenotype. In some embodiments, the phenotype may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after contacting the cell exhibiting the phenotype with the potential drug candidate.

In some embodiments, the phenotype may include gene expression patterns of a cell affected by a neurodegenerative disease or disorder. In some embodiments, the phenotype may include, but is not limited to, activation and/or expression of ERK, NFKB, p38 MAPK, or MCP-1. In some embodiments, the activation and/or expression of ERK, NFKB, p38 MAPK, or MCP-1 may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after contacting the cell exhibiting the phenotype with the potential drug candidate.

In some embodiments, the phenotype may include formation of reactive oxygen species. Reactive oxygen species may include, but are not limited to, superoxide (O₂^{·-}), hydrogen peroxide (H₂O₂), nitric oxide (NO), peroxynitrite (ONOO⁻), hypochlorous acid (HOCl), hypobromous acid (HOBr), hydroxyl radical (HO^{·}), peroxy radical (ROO^{·}), alkoxy radical (RO^{·}), singlet oxygen (¹O₂) and combinations thereof. In some embodiments, the reactive oxygen species may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after contacting the cell exhibiting the phenotype with the potential drug candidate.

In some embodiments, the method further includes measuring the biological age of the subject. In some embodiments, measuring the biological age of the subject is determining the chronological age of the subject and reversing or decreasing the rate of increase in the biological age of the subject, thereby preventing a disease or condition in the subject. In some embodiments, the method further includes administering the drug candidate in combination with other therapies. In some embodiments, the method further includes tailoring the drug administration based on individual genetic profiles or biological age markers. In some embodiments, the method further includes administering the drug candidate as a prevention measure in subjects at high risk of developing age-related disease. In some embodiments, the method further includes assessing the long-term effects of the drug candidate on biological age markers and disease progression. In some embodiments, the method further includes using other biological age markers such as telomere length, mitochondrial function, or protein aggregation levels. In some embodiments, the method further includes developing non-invasive methods to measure biological age and monitor disease progression, like blood tests or imaging techniques. In some embodiments, the method further includes measuring cognitive function or neurological health as part of the evaluation of the drug candidate's efficacy. In some embodiments, the method further includes considering the impact of lifestyle factors (for example, diet, exercise, etc.) on biological age and treatment efficacy. In some embodiments, the method further includes evaluating different dosing regimens to determine the most effective and safe protocol. In some embodiments, the method further includes evaluating different dosing regimens to determine effective and safe protocols. In some embodiments, the method further includes developing formulations of the drug candidate that are tailored to different age groups or stages of disease progression.

In some embodiments, the method may further include validating the responsiveness of the drug candidate in an in vivo model. In some embodiments, the in vivo model may be selected from, but not limited to, rodent models (for example, mice and rats), genetically modified models, disease induced models, rabbit models, zebrafish models, non-human primate models, chicken embryo models, fruit fly models, nematode models, xenograft models (for example, human tumor cells in mice), or a combination thereof.

In some embodiments, the method further includes using artificial intelligence or machine learning algorithms to predict the efficacy of the potential drug candidate. In some embodiments, the artificial intelligence or machine learning algorithms to predict the efficacy of potential drug candidates may include a commercially available platform. A non-exhaustive list of examples of artificial intelligence or machine learning algorithms platforms include IBM Watson for Drug Discovery, Atomwise, BenevolentAI, Insilico Medicine, Deep Genomics, Recursion Pharmaceutics, Excientia, BioSymetrics, Nuedii, TwoZAR, Cloud Pharmaceuticals, VeriSIM Life, Schrodinger, and GNS Healthcare.

In some embodiments, the method further includes analyzing a potential drug candidates based on genetic, epigenetic, or metabolomics of a selected population. In some embodiments, the method further includes dosing of the potential drug candidates based on genetic, epigenetic, or metabolomics of a subject. In some embodiments the genetic, epigenetic, or metabolomics may include genetic profiling. For example, a genotype to identify a common genetic mutation. In some embodiments the genetic, epigenetic, or metabolomics may include epigenetic analysis in neurodegenerative diseases. For example, a subject with early-stage Alzheimer's disease may be selected to undergo epigentic profiling. In some embodiments, histone modification patterns and DNA methylation levels may be analyzed. In some embodiments, the potential drug candidate may be tested based on their ability to modulate these epigenetic marks, potentially reversing or slowing the progression of the disease. In some embodiments the genetic, epigenetic, or metabolomics may include metabolomic screening for cardiovascular or neurological diseases. For example, a population with a high risk of cardiovascular diseases may be subjected to metabolomic analysis to identify unique metabolic signatures linked to a neurological or cardiovascular risk. In some embodiments, a potential drug candidate may be assessed for their impact on these metabolomic profiles.

In some embodiments, the method further includes the step of gene editing the sample to introduce or correct one or more mutations associated with the disease or condition. In some embodiments, the disease or condition is associated with a biological clock. In some embodiments, the gene editing may include using CRISPR-Cas9 or a related CRISPR technology for gene editing. In some embodiments, the gene editing may introduce a mutation into the sample. In some embodiments, the mutation may provide the ability to correct the pathological effects of a disease or condition. In some embodiments, the mutation may provide a model for study of the mutation and the potential drug candidate. For example, genes such as APP, PSEN1, or PSEN2 in neuronal cells may be edited to model familial Alzheimer's disease. In some embodiments, the gene edited sample may be used to in the in vitro analysis. In some embodiments, the gene edited sample may be used in combination with the potential drug candidate to reduce amyloid-beta production or aggregation.

In some embodiments, the potential drug candidate may be further evaluated for toxicity, side effects, or drug to drug interactions. In some embodiments, the potential drug candidate may be formulated with one or more additional active ingredients to enhance therapeutic efficacy. In some embodiments, the method may further include administering the potential drug candidate in combination with other therapeutic agents for a synergistic or additive effect.

In some embodiments, the method further includes using computational modeling to predict the interaction between the potential drug candidate and the target associated with the biological clock. In some embodiments, the computation modeling may be a molecular docking simulation. In some embodiments, the docking simulation may predict how different small molecules interact with key proteins in the subject or sample. In some embodiments, the computational modeling may be applied to existing drug libraries to identify compounds the could bind to and modulate one or more desired biological targets. In some embodiments, the computational modeling may be in silico models. In some embodiments, the in silico models may predict how potential drug candidates interact with amyloid-beta or tau proteins.

In some embodiments, the method further includes a step of using 3D cell culture techniques. In some embodiments, the method further includes a step of using organ-on-a-chip models. In some embodiments, incorporating 3D cell culture techniques or organ-on-a-chip models into the drug screening process may offer a more physiologically relevant environment for evaluating potential drug candidate. In some embodiments, a cultivate brain organoids from induced pluripotent stem cells may be used to test drugs targeting neurological disorders like Alzheimer's or Parkinson's disease. In some embodiments, brain organoids may offer a complex, multi-cell type environment that closely resembles actual brain tissue.

In some embodiments, the disease or condition associated with biological clocks in the subject in need thereof is based on modulation of DNA methylation of genes associated with biological clocks. In some embodiments, the disease or condition associated with a biological clock in a subject in need thereof is associated with genes or genomic regions hypermethylated with age. In some embodiments, the disease or condition associated with a biological clock in a subject in need thereof is associated with genes or genomic regions hypomethylated with age. In some embodiments, the disease or condition associated with a biological clock in a subject in need thereof is associated with Tau phosphorylation. In some embodiments, the disease or condition associated with a biological clock in a subject in need thereof is associated with hyperglycemia. In some embodiments, the disease or condition associated with a biological clock in a subject in need thereof is associated with hyperinsulinemia. In some embodiments, the disease or condition associated with a biological clock in a subject in need thereof is associated with epigenetic age reversal. In some embodiments, the disease or condition associated with a biological clock in a subject in need thereof is associated with oxidative stress reduction. In some embodiments, the disease or condition associated with a biological clock in a subject in need thereof is associated with neuroprotection. In some embodiments, the disease or condition associated with a biological clock in a subject in need thereof is associated with cardiovascular health improvement. In some embodiments, the disease or condition associated with a biological clock in a subject in need thereof is associated with mitochondrial function optimization. In some embodiments, the disease or condition associated with a biological clock in a subject in need thereof is associated with musculoskeletal health. In some embodiments, the disease or condition associated with a biological clock in a subject in need thereof is associated with cognitive function enhancement. In some embodiments, the disease or condition associated with a biological clock in a subject in need thereof is associated with personalized medicine. In some embodiments, the disease or condition associated with a biological clock in a subject in need thereof is associated with lifestyle intervention integration. In some embodiments, the disease or condition associated with a biological clock in a subject in need thereof is associated with biomarker monitoring.

In some embodiments, the drug candidate is provided to a subject to treat, prevent, reduce, or ameliorate a disease a condition. In some embodiments, the subject may experience an improvement in symptoms or conditions related to genes or genomic regions hypermethylated with age after administration of a drug candidate as described herein. In some embodiments, the improvement in symptoms related to genes or genomic regions hypermethylated with age may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate. For example, subjects may an improvement in symptoms or conditions related to genes or genomic regions hypermethylated with age ranging from approximately 5% to 100% after administration of the drug candidate and at least one pharmaceutically acceptable excipient.

In some embodiments, the subject may experience an improvement in symptoms or conditions related to genes or genomic regions hypomethylated with age after administration of a drug candidate as described herein. In some embodiments, the subject may experience an improvement in symptoms or conditions related to genes or genomic regions hypomethylated with age after administration of a drug candidate as described herein. In some embodiments, the improvement in symptoms related to genes or genomic regions hypomethylated with age may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may an improvement in symptoms or conditions related to genes or genomic regions hypomethylated with age ranging from approximately 5% to 100% after administration of the drug candidate and at least one pharmaceutically acceptable excipient.

In some embodiments, the subject may experience an improvement in symptoms or conditions related to hyperglycemia (which can lead to type I and type II diabetes) after administration of a drug candidate as described herein. In some embodiments, the subject may experience an improvement in symptoms or conditions related to hyperglycemia (which can lead to type I and type II diabetes) after administration of the drug candidate. In some embodiments, the improvement in symptoms related to hyperglycemia may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a drug candidate as described herein. For example, subjects may an improvement in symptoms or conditions related to hyperglycemia ranging from approximately 5% to 100% after administration of the drug candidate and at least one pharmaceutically acceptable excipient.

In some embodiments, the subject may experience an improvement in symptoms or conditions related to hyperlipidemia (such as obesity-related conditions) after administration of a drug candidate as described herein. In some embodiments, the subject may experience an improvement in symptoms or conditions related to hyperlipidemia (such as obesity-related conditions) after administration of the drug candidate. In some embodiments, the improvement in symptoms related to hyperlipidemia may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate and at least one pharmaceutically acceptable excipient. For example, subjects may an improvement in symptoms or conditions related to hyperlipidemia ranging from approximately 5% to 100% after administration of the drug candidate and at least one pharmaceutically acceptable excipient.

In some embodiments, the subject may experience an improvement in symptoms or conditions related to hyperinsulinemia after administration of a drug candidate as described herein. In some embodiments, the subject may experience an improvement in symptoms or conditions related to hyperinsulinemia after administration of the drug candidate. In some embodiments, the improvement in symptoms related to hyperinsulinemia may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may an improvement in symptoms or conditions related to hyperinsulinemia ranging from approximately 5% to 100% after administration of the drug candidate and at least one pharmaceutically acceptable excipient.

In some embodiments, the subject may experience a reduction or decrease in symptoms related to an Alzheimer's Disease Composite Score after administration of a drug candidate as described herein. In some embodiments, the subject may experience a reduction or decrease in symptoms related to an Alzheimer's Disease Composite Score after administration of the drug candidate. In some embodiments, the reduction or decrease in symptoms related to an Alzheimer's Disease Composite Score may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to Alzheimer's Disease Composite Score ranging from approximately 5% to 100% after administration of the drug candidate and at least one pharmaceutically acceptable excipient. In some embodiments, reducing or decreasing symptoms related to Alzheimer's Disease Composition Score may reduce other neuropsychological measures, including but not limited to, ADAS-Cog, MoCA, MMSE, CDR, QDRS, PDQ-9. In some embodiments, reducing or decreasing symptoms related to Alzheimer's Disease Composition Score may improve neuroimaging, including but not limited to, ASL, BOLD, MRS, task-based or resting fMRI, vMRI and FDG-PET.

In some embodiments, the subject may experience a reduction or decrease in symptoms related to CSF phosphorylated tau (“pTau”) after administration of a drug candidate as described herein. In some embodiments, the subject may experience a reduction or decrease in symptoms related to pTau after administration of the drug candidate. In some embodiments, the reduction or decrease in symptoms related to pTau may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to pTau ranging from approximately 5% to 100% after administration of the drug candidate and at least one pharmaceutically acceptable excipient.

In some embodiments, the subject may experience a reduction or decrease in symptoms related to leptin deficiency after administration of a drug candidate as described herein. In some embodiments, the subject may experience a reduction or decrease in symptoms related to leptin deficiency after administration of the drug candidate. In some embodiments, the reduction or decrease in symptoms related to leptin deficiency may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to leptin deficiency ranging from approximately 5% to 100% after administration of the drug candidate and at least one pharmaceutically acceptable excipient.

In some embodiments, the subject may experience a reduction or decrease in symptoms related to leptin resistance after administration of a drug candidate as described herein. In some embodiments, the subject may experience a reduction or decrease in symptoms related to leptin resistance after administration of the drug candidate. In some embodiments, the reduction or decrease in symptoms related to leptin resistance may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to leptin resistance ranging from approximately 5% to 100% after administration of the drug candidate and at least one pharmaceutically acceptable excipient.

In some embodiments, the subject may experience a reduction or decrease in symptoms related to DNA methylation after administration of a drug candidate as described herein. In some embodiments, the subject may experience a reduction or decrease in symptoms related to DNA methylation after administration of the drug candidate. In some embodiments, the reduction or decrease in symptoms related to DNA methylation may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate and at least one pharmaceutically acceptable excipient. For example, subjects may experience reduction in symptoms related to DNA methylation ranging from approximately 5% to 100% after administration of the drug candidate and at least one pharmaceutically acceptable excipient.

In some embodiments, the subject may experience a reduction or decrease in symptoms related to cardiovascular risk after administration of a drug candidate as described herein. In some embodiments, the subject may experience a reduction or decrease in symptoms related to cardiovascular risk after administration of the drug candidate. In some embodiments, the reduction or decrease in symptoms related to cardiovascular risk may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to cardiovascular risk ranging from approximately 5% to 100% after administration of the drug candidate and at least one pharmaceutically acceptable excipient.

In some embodiments, the subject may experience a reduction or decrease in symptoms related to a subject's epigenetic age (“DNA methylation phenoage”) after administration of a drug candidate as described herein. In some embodiments, the subject may experience a reduction or decrease in symptoms related to DNA methylation phenoage after administration of the drug candidate. In some embodiments, the reduction or decrease in symptoms related to DNA methylation phenoage may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to DNA methylation phenoage ranging from approximately 5% to 100% after administration of the drug candidate and at least one pharmaceutically acceptable excipient.

In some embodiments, the subject may experience a reduction or decrease in symptoms related to the subject's epigenetic clock (“DNA methylation skin blood clock”) after administration of a drug candidate as described herein. In some embodiments, the subject may experience a reduction or decrease in symptoms related to the subject's DNA methylation skin blood clock after administration of 17a-ethynylandrost-5-ene-3B,7B,17B-triol. In some embodiments, the reduction or decrease in symptoms related to DNA methylation skin blood clock may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to DNA methylation skin blood clock ranging from approximately 5% to 100% after administration of the drug candidate and at least one pharmaceutically acceptable excipient.

In some embodiments, the subject may experience a reduction or decrease in symptoms related to the subject's DunedinPACE clock after administration of a drug candidate as described herein. In some embodiments, the subject may experience a reduction or decrease in symptoms related to the subject's DunedinPACE clock after administration of the drug candidate. In some embodiments, the reduction or decrease in symptoms related to DunedinPACE clock may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to DunedinPACE clock ranging from approximately 5% to 100% after administration of the drug candidate and at least one pharmaceutically acceptable excipient.

In some embodiments, the subject may experience a prevention, reduction or decrease in conditions or symptoms related to a cancer or tumor after administration of a drug candidate as described herein. In some embodiments, the subject may experience a prevention, reduction or decrease in conditions or symptoms related to a cancer or tumor after administration of the drug candidate. In some embodiments, the prevention, reduction or decrease in conditions or symptoms related to a cancer or tumor may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience prevention, reduction or decrease in conditions or symptoms related to a cancer or tumor ranging from approximately 5% to 100% after administration of 17a-ethynylandrost-5-ene-3B,7B,17B-triol and at least one pharmaceutically acceptable excipient.

In some embodiments, the subject may experience a reduction or decrease in symptoms related to atherosclerosis after administration of a drug candidate as described herein. In some embodiments, the subject may experience a reduction or decrease in symptoms related to atherosclerosis after administration of the drug candidate. In some embodiments, the reduction or decrease in symptoms related to atherosclerosis may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to atherosclerosis ranging from approximately 5% to 100% after administration of the drug candidate and at least one pharmaceutically acceptable excipient.

In some embodiments, the subject may experience a reduction or decrease in symptoms related to schizophrenia after administration of a drug candidate as described herein. In some embodiments, the subject may experience a reduction or decrease in symptoms related to schizophrenia after administration of the drug candidate. In some embodiments, the reduction or decrease in symptoms related to schizophrenia may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to schizophrenia ranging from approximately 5% to 100% after administration of the drug candidate and at least one pharmaceutically acceptable excipient.

In some embodiments, the subject may experience a reduction or decrease in symptoms related to an autoimmune disease after administration of a drug candidate as described herein. In some embodiments, the subject may experience a reduction or decrease in symptoms related to an autoimmune disease after administration of the drug. In some embodiments, the reduction or decrease in symptoms related to an autoimmune disease may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of a composition as described herein. For example, subjects may experience reduction in symptoms related to an autoimmune disease ranging from approximately 5% to 100% after administration of the drug candidate and at least one pharmaceutically acceptable excipient.

In some embodiments, the subject may experience a reduction or decrease in symptoms related to rheumatoid arthritis after administration of a drug candidate as described herein. In some embodiments, the subject may experience a reduction or decrease in symptoms related to rheumatoid arthritis after administration of the drug candidate. In some embodiments, the reduction or decrease in symptoms related to rheumatoid arthritis may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to rheumatoid arthritis ranging from approximately 5% to 100% after administration of the drug candidate and at least one pharmaceutically acceptable excipient.

In some embodiments, the subject may experience a reduction or decrease in symptoms related to systemic lupus erythematosus after administration of a drug candidate as described herein. In some embodiments, the subject may experience a reduction or decrease in symptoms related to systemic lupus erythematosus after administration of the drug candidate. In some embodiments, the reduction or decrease in symptoms related to systemic lupus erythematosus may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to systemic lupus erythematosus ranging from approximately 5% to 100% after administration of the drug candidate and at least one pharmaceutically acceptable excipient.

In some embodiments, the subject may experience a reduction or decrease in symptoms related to multiple sclerosis after administration of a drug candidate as described herein. In some embodiments, the subject may experience a reduction or decrease in symptoms related to multiple sclerosis after administration of the drug candidate. In some embodiments, the reduction or decrease in symptoms related to multiple sclerosis may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience reduction in symptoms related to multiple sclerosis ranging from approximately 5% to 100% after administration of the drug candidate and at least one pharmaceutically acceptable excipient.

In some embodiments, the subject may experience prevention or a reduction or decrease in the risk to ovum or sperm related to increased risks of offspring with autism spectrum disorder after administration of a drug candidate as described herein. In some embodiments, the subject may experience prevention or a reduction or decrease in risk to ovum or sperm related to increased risks of offspring with symptoms related to autism spectrum disorder after administration of the drug candidate. In some embodiments, the reduction or decrease in ovum or sperm with increased risks of an offspring with symptoms related to autism spectrum disorder may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience prevention, reduction or decrease in ovum or sperm with increased risks of an offspring with symptoms related to autism spectrum disorder ranging from approximately 5% to 100% after administration of the drug candidate and at least one pharmaceutically acceptable excipient.

In some embodiments, the subject may experience prevention or a reduction or decrease in ovum or sperm with increased risks of an offspring with Down Syndrome after administration of a drug candidate as described herein. In some embodiments, the subject may experience prevention or a reduction or decrease in ovum or sperm with increased risks of an offspring with Down Syndrome after administration of the drug candidate. In some embodiments, the reduction or decrease in symptoms related to Down Syndrome may be reduced by an amount equal to or greater than approximately 5%, 10%, 15%, 20%, 25%, 30%, 35%, 40%, 45%, 50%, 55%, 60%, 65%, 70%, 75%, 80%, 85%, 90%, 99%, 100%, or ranges including and/or spanning the aforementioned values after administration of the drug candidate as described herein. For example, subjects may experience prevention or reduction in ovum or sperm with increased risks of an offspring with Down Syndrome ranging from approximately 5% to 100% after administration of the drug candidate and at least one pharmaceutically acceptable excipient.

Accordingly, some aspects described herein relate to the following numbered alternatives:

1. An in vitro screening method to identify a potential drug candidate capable of treating, preventing, reducing, or ameliorating a disorder or disease, including: (i) providing a sample for stimulation selected from the group consisting of a cell, tissue, blood, monocytes, microglia, macrophages, adipocytes, neuroblastoma, pheochromocytoma, and Lund human mesencephalic (LUHMES) cells; (ii) stimulating the sample with an agonist to induce a phenotype or phenotypic reaction, wherein the phenotype or phenotypic reaction substantially corresponds to a disease or condition associated with a biological clock; (iii) contacting the sample exhibiting the phenotype or phenotypic reaction with one or more potential drug candidates; (iv) determining a responsive change in the phenotype or phenotypic reaction of the sample; and (v) providing the one or more potential drug candidates to a subject to treat, reduce, prevent, or ameliorate a disease or condition associated with a biological clock in a subject.

2. The method of alternative 1, wherein the responsive change is a decrease or loss in the phenotype and the decrease or loss is indicative that the potential drug candidate is capable of preventing, reducing, or ameliorating a neurodegenerative disorder or disease.

3. The method of alternative 1, wherein the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism.

4. The method of alternative 1 or 2, wherein the neurodegenerative disorder or disease is Parkinson's disease.

5. The method of alternative 1 or 2, wherein the neurodegenerative disorder is Alzheimer's disease.

6. The method of any one of alternatives 1 to 4, wherein the disease or condition associated with biological clocks in the subject in need thereof is based on modulation of DNA methylation of genes associated with biological clocks.

7. The method of alternative 5, wherein the disease or condition associated with the biological clock in the subject in need thereof is associated with genes or genomic regions hypermethylated with age.

8. The method of alternative 5, wherein the disease or condition associated with a biological clock in the subject in need thereof is associated with genes or genomic regions hypomethylated with age.

9. The method of alternative 5, wherein the disease or condition associated with inflammatory TNF signaling.

10 The method of alternative 5, wherein the disease or condition associated with inflammatory NF-kB signaling.

11. The method of any one of alternatives 1 to 7, wherein the disease or condition associated with a biological clock in the subject in need thereof is associated with Tau phosphorylation.

12. The method of any one of alternatives 1 to 7, wherein the disease or condition associated with a biological clock in the subject in need thereof is associated with hyperglycemia.

13. The method of any one of alternatives 1 to 7, wherein the disease or condition associated with the biological clock in the subject in need thereof is associated with hyperinsulinemia.

14. The method of any one of alternatives 1 to 7, wherein the disease or condition associated with the biological clock in the subject in need thereof is associated with obesity.

15. The method of any one of alternatives 1 to 7, wherein the disease or condition associated with the biological clock in the subject is connected to leptin.

16. The method of alternative 14, wherein the subject experiences a decrease between about a 5% to about a 100% reduction in conditions or symptoms connected to leptin after administration of the one or more drug candidate and at least one pharmaceutically acceptable excipient.

16. The method of any one of alternatives 1 to 7, wherein the disease or condition associated with a biological clock is associated with a subject's SkinBloodAge.

17. The method of alternative 16, wherein the subject experiences a decrease between about a 5% to about a 100% reduction in conditions or symptoms connected to SkinBloodAge after administration of the one or more drug candidate and at least one pharmaceutically acceptable excipient.

18. The method of any one of alternatives 1 to 7, wherein the disease or condition associated with the biological clock is associated with a subject's dinucleotide (CpG) methylation in association with leptin promotor (DNAmLeptin).

19. The method of alternative 18, wherein the subject experiences a decrease between about a 5% to about a 100% reduction in conditions or symptoms connected to DNAmLeptin after administration of the drug candidate and at least one pharmaceutically acceptable excipient.

20. The method of any one of alternatives 1 to 7, wherein the disease or condition associated with a biological clock is associated with a subject's DNAmPack Years.

21. The method of alternative 20, wherein the subject experiences a decrease between about a 5% to about a 100% reduction in conditions or symptoms connected to DNAmPackYears after administration of the drug candidate and at least one pharmaceutically acceptable excipient.

22. A method to treat, prevent, reduce, or ameliorate a neurodegenerative disorder or disease, including: (i) providing a sample for stimulation selected from the group consisting of a cell, tissue, blood, monocytes, microglia, macrophages, adipocytes, neuroblastoma, pheochromocytoma, and Lund human mesencephalic (LUHMES) cells; (ii) stimulating the sample with an agonist to induce a phenotype or phenotypic reaction, wherein the phenotype or phenotypic reaction substantially corresponds to a disease or condition associated with a biological clock; (iii) contacting the sample exhibiting the phenotype or phenotypic reaction with one or more potential drug candidates; (iv) determining a responsive change in the phenotype or phenotypic reaction of the sample; and (v) providing the one or more potential drug candidates to a subject to treat, reduce, prevent, or ameliorate a disease or condition associated with a biological clock in a subject.

23. The method of alternative 22, wherein the responsive change is a decrease or loss in the phenotype and the decrease or loss is indicative that the potential drug candidate is capable of preventing, reducing, or ameliorating a neurodegenerative disorder or disease.

24. The method of alternative 23, wherein the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism.

25. The method of alternative 22 or 23, wherein the neurodegenerative disorder or disease is Parkinson's disease.

26. The method of alternative 22 or 23, wherein the neurodegenerative disorder is Alzheimer's disease.

27. The method of any one of alternatives 22 to 26, wherein the disease or condition associated with biological clocks in the subject in need thereof is based on modulation of DNA methylation of genes associated with biological clocks.

28. The method of alternative 27, wherein the disease or condition associated with the biological clock in the subject in need thereof is associated with genes or genomic regions hypermethylated with age.

29. The method of alternative 27, wherein the disease or condition associated with the biological clock in the subject in need thereof is associated with genes or genomic regions hypomethylated with age.

30. The method of alternative 27, wherein the disease or condition associated with inflammatory TNF signaling.

31. The method of alternative 27, wherein the disease or condition associated with inflammatory NF-kB signaling.

32. The method of any one of alternatives 22 to 29, wherein the disease or condition associated with the biological clock in a subject in need thereof is associated with Tau phosphorylation.

33. The method of any one of alternatives 22 to 29, wherein the disease or condition associated with the biological clock in a subject in need thereof is associated with hyperglycemia.

34. The method of any one of alternatives 22 to 29, wherein the disease or condition associated with the biological clock in a subject in need thereof is associated with hyperinsulinemia.

35. The method of any one of alternatives 22 to 29, wherein the disease or condition associated with the biological clock in a subject in need thereof is associated with obesity.

36. The method of any one of alternatives 22 to 29, wherein the disease or condition associated with the biological clock in a subject is connected to leptin.

37. The method of alternative 36, wherein the subject experiences a decrease between about a 5% to about a 100% reduction in conditions or symptoms connected to leptin after administration of the drug candidate and at least one pharmaceutically acceptable excipient.

38. The method of any one of alternatives 22 to 29, wherein the disease or condition associated with the biological clock is associated with a subject's SkinBloodAge.

39. The method of alternative 38, wherein the subject experiences a decrease between about a 5% to about a 100% reduction in conditions or symptoms connected to SkinBloodAge after administration of the drug candidate and at least one pharmaceutically acceptable excipient.

40. The method of any one of alternatives 22 to 29, wherein the disease or condition associated with a biological clock is associated with a subject's dinucleotide (CpG) methylation in association with leptin promotor (DNAmLeptin).

41. The method of alternative 40, wherein the subject experiences a decrease between about a 5% to about a 100% reduction in conditions or symptoms connected to DNAmLeptin after administration of the drug candidate and at least one pharmaceutically acceptable excipient.

42. The method of any one of alternatives 22 to 29, wherein the disease or condition associated with a biological clock is associated with a subject's DNAmPack Years.

43. The method of alternative 42, wherein the subject experiences a decrease between about a 5% to about a 100% reduction in conditions or symptoms connected to DNAmPackYears after administration of the drug candidate and at least one pharmaceutically acceptable excipient.

44. The method of any one of alternatives 1 to 43, wherein the sample for stimulation further includes cells selected from induced pluripotent stem cells, mesenchymal stem cells, or endothelial cells.

45. The method of any one of alternatives 1 to 44, wherein the agonist used for stimulation is selected from a group consisting of cytokines, chemokines, growth factors, or environmental stressors like oxidative stress or hypoxic conditions.

46. The method of any one of alternatives 1 to 45, wherein the potential drug candidate is selected from the group consisting of small molecules, peptides, antibodies, RNA-based therapies, or gene-editing tools.

47. The method of any one of alternatives 1 to 46, wherein the determining of a responsive change in phenotype or phenotypic reaction includes measuring changes in cellular metabolism, morphology, or signaling pathways.

48. The method of alternative 47, wherein the change in cellular signaling pathways includes modulation of pathways associated with autophagy, apoptosis, or senescence.

49. The method of any one of alternatives 1 to 48, wherein the disease or condition associated with the biological clock includes conditions related to cardiovascular health, such as atherosclerosis or hypertension.

50. The method of any one of alternatives 1 to 49, wherein the disease or condition associated with the biological clock includes conditions related to bone health, such as osteoporosis or osteoarthritis.

51. The method of any one of alternatives 1 to 50, further comprising validating the responsiveness of the drug candidate in an in vivo model.

52. The method of any one of alternatives 1 to 51, wherein the potential drug candidate is further evaluated for toxicity, side effects, or drug-drug interactions.

53. The method of any one of alternatives 1 to 52, further comprising formulating the drug candidate with one or more additional active ingredients to enhance therapeutic efficacy.

54. The method of any one of alternatives 1 to 53, further comprising administering the drug candidate in combination with other therapeutic agents for a synergistic or additive effect.

55. The method of any one of alternatives 1 to 54, wherein the sample for stimulation is derived from a subject known to have or be at risk for the disease or condition associated with the biological clock.

56. The method of any one of alternatives 1 to 55, wherein the improvement in symptoms or conditions is additionally measured by changes in the quality of life, physical mobility, or cognitive function of the subject.

57. The method of alternative 56, wherein the changes in cognitive function are assessed using standardized neuropsychological tests.

58. The method of any one of alternatives 1 to 57, further comprising using artificial intelligence or machine learning algorithms to predict the efficacy of the potential drug candidate.

59. The method of any one of alternatives 1 to 58, further comprising including dosing of the drug candidate based on genetic, epigenetic, or metabolomic profiling of the subject.

60. The method of any one of alternatives 1 to 59, wherein the disease or condition associated with a biological clock includes mental health disorders, such as depression, bipolar disorder, or anxiety disorders.

61. The method of any one of alternatives 1 to 60, wherein the determination of a responsive change in the phenotype or phenotypic reaction further includes the use of imaging techniques, such as fluorescence microscopy or magnetic resonance imaging (MRI).

62. The method of any one of alternatives 1 to 61, wherein the sample for stimulation includes cells derived from specific organ systems, such as hepatic, renal, or pulmonary cells, for targeted disease modeling.

63. The method of any one of alternatives 1 to 62, wherein the one or more potential drug candidates are identified using a high-throughput screening method involving automated robotic systems.

64. The method of any one of alternatives 1 to 63, further comprising the step of gene editing the sample cells to introduce or correct mutations associated with the disease or condition related to the biological clock.

65. The method of any one of alternatives 1 to 64, wherein the potential drug candidate is formulated in a sustained-release or controlled-release dosage form for prolonged therapeutic effect.

66. The method of any one of alternatives 1 to 65, wherein the sample for stimulation is derived from a biobank of diverse genetic backgrounds to ensure broad applicability of the drug candidate.

67. The method of any one of alternatives 1 to 66, wherein the potential drug candidate is evaluated for its ability to cross the blood-brain barrier for treatment of neurological disorders.

68. The method of any one of alternatives 1 to 67, wherein the one or more potential drug candidates are tested for their potential to reverse age-related epigenetic changes.

69. The method of any one of alternatives 1 to 68, wherein the disease or condition associated with the biological clock includes autoimmune disorders, such as rheumatoid arthritis or lupus.

70. The method of any one of alternatives 1 to 69, further comprising a step for individualized treatment planning based on the genomic and proteomic analysis of the subject.

71. The method of any one of alternatives 1 to 70, wherein the responsive change in the phenotype or phenotypic reaction is quantified using a scoring system based on a set of predefined criteria.

72. The method of any one of alternatives 1 to 71, further comprising the use of telemedicine or digital health platforms for monitoring the subject's response to the drug candidate post-administration.

73 The method of any one of alternatives 1 to 72, wherein the potential drug candidate is further evaluated for its ability to modulate microbiome composition as part of its therapeutic action.

74. The method of any one of alternatives 1 to 73, wherein the method further includes the use of a biomarker panel for monitoring the progression of the disease or condition associated with the biological clock.

75. The method of any one of alternatives 1 to 74, wherein the disease or condition associated with a biological clock includes dermatological conditions, such as psoriasis, eczema, or aging-related skin changes.

76. The method of any one of alternatives 1 to 75, further comprising a step of using computational modeling to predict the interaction between the potential drug candidate and the target associated with the biological clock.

77. The method of any one of alternatives 1 to 76, wherein the sample for stimulation is subjected to genetic profiling to identify mutations or alterations associated with the disease or condition related to the biological clock.

78. The method of any one of alternatives 1 to 77, wherein the disease or condition associated with a biological clock includes gastrointestinal disorders, such as inflammatory bowel disease, irritable bowel syndrome, or colorectal cancer.

79. The method of any one of alternatives 1 to 78, wherein the agonist used for stimulation is a pathogen or pathogen-derived molecule to simulate infection-related conditions associated with a biological clock.

80. The method of any one of alternatives 1 to 79, wherein the potential drug candidate is a combination therapy comprising multiple active ingredients targeting different aspects of the biological clock.

81. The method of any one of alternatives 1 to 80, wherein the sample for stimulation includes cells from the immune system, such as lymphocytes or dendritic cells, to study immune-related disorders associated with a biological clock.

82. The method of any one of alternatives 1 to 81, further comprising a step of using 3D cell culture techniques or organ-on-a-chip models for more physiologically relevant drug screening.

83. The method of any one of alternatives 1 to 82, wherein the method further includes the use of next-generation sequencing technologies to assess the impact of the drug candidate on the genetic and epigenetic landscape of the sample.

84. The method of any one of alternatives 1 to 83, wherein the potential drug candidate is screened for its ability to modulate circadian rhythms in the subject.

85. The method of any one of alternatives 1 to 84, wherein the disease or condition associated with a biological clock includes respiratory disorders, such as asthma, chronic obstructive pulmonary disease (COPD), or lung fibrosis.

86. The method of any one of alternatives 1 to 85, further comprising a step of evaluating the environmental and lifestyle factors of the subject that may influence the efficacy of the drug candidate.

87. The method of any one of alternatives 1 to 86, wherein the potential drug candidate is evaluated for its effects on cellular aging processes, such as telomere length or mitochondrial function.

88. The method of any one of alternatives 1 to 87, wherein the method includes a step of assessing the pharmacokinetics and pharmacodynamics of the drug candidate in various biological matrices.

89. The method of any one of alternatives 1 to 88, wherein the disease or condition associated with a biological clock includes endocrine disorder.

### EXAMPLES

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. Changes therein and other uses which are encompassed within the spirit of the disclosure as defined by the scope of the claims will occur to those skilled in the art.

### Example 1. Treatment Induced Epigenetic Modifications in MCI and Mild Probable AD

NE3107 is an oral small molecule, blood-brain permeable anti-inflammatory insulin sensitizer that binds ERK, inhibits NFKB and TNF signaling, and is in clinical studies for Probable AD (NCT04669028) and Parkinson's disease (NCT05083260). Safety, clinical, biomarker and neuroimaging results from a 3-month open-label phase 2 study of NE3107 treatment of MCI and probable AD (NCT05007820) were reported at CTAD (2022 J. Prev. Alzheimers Dis 9 S86, S140 and S27). In this example, the inventors determined the results of epigenetic clock studies from NCT05007820.

Methods: Subjects with clinical dementia rating (CDR) 0.5-1 were consented, and whole blood was drawn, frozen and shipped for Horvath Clock DNA methylation (DNAm) epigenetic analysis (Horvath 2018 Aging 10:1758) at baseline and 3 months.

Result: The subject's SkinBloodAge clock closely matched their chronological age at baseline (mean 71.5 vs 70.8 years). After 3 months of NE3107 (20 mg BID), the SkinBloodAge decreased by a mean of 3.3 years (Table). The biologic age decreased significantly in 86% of subjects at 3 months. Dinucleotide (CpG) methylation in association with the leptin promotor (DNAmLeptin) was significantly decreased (−40%) in 100% of subjects. Leptin is reportedly lower in AD, and DNAmLeptin is reported to be positively correlated with metabolic parameters waist/hip ratio, BMI, CRP, insulin, glucose and triglycerides (Lu 2022 Aging 14:9884). DNAmPackYears also significantly decreased in 95% by 39%. DNAmPack Years is reported to be negatively correlated with time to mortality in both smokers and nonsmokers, and positively correlated with waist/hip ratio, CRP and triglycerides. Monocyte DNAm was correlated with monocyte frequency at baseline, and although it is reported to increases with chronological age, it decreased (−29%) in 77% of subjects after 3 months treatment. There was no significant change in monocyte frequency after 3 months. There were no significant changes in DNAmT, DNAmNK or DNAmGran. The results are described in Table 1.

TABLE 1

| | | | Change | | | Frequency |
|---|---|---|---|---|---|---|
| | Baseline | 3 Months | (%) | t-test p | 95% CI | (%) |
| Cronologic Age | 71.5 | 71.8 | 0.28 | — | — | — |
| (Yrs) | (9.3)¹ | (9.3) | (4.6%) | | | |
| DNAmSkinBlood | 70.8 | 67.5 | −3.3 | 0.002 | −5.3 to | 19/22 |
| | (10.2) | (8.3) | (−4.6%) | | −1.4 | (86%) |
| DNAmPackYears | 24.1 | 14.6 | −9.4 | 0.0005 | −14.2 to | 21/22 |
| | (9.3) | (−9.3) | (39%) | | −4.6 | (95%) |
| DNAmLeptin | 13141 | 7829 | −5312 | <0.0001 | −6727 to | 22/22 |
| (CpGs) | (3634) | (3595) | (−40%) | | −3897 | (100%) |
| Blood Cell | | | | | | |
| Frequency | | | | | | |
| DNAmMonocytes | 0.076 | 0.054 | −0.022 | 0.001 | −0.034 to | 17/22 |
| (Freq) | (0.019) | (0.024) | (29%) | | −0.0102 | (77%) |
| Monocytes (Freq) | 0.085 | 0.085 | −0.001 | ns | — | — |
| | (0.021) | (0.023) | (1.5%) | | | |
| Correlation² | 0.62 | 0.42 | 0.16 | | | |
| | (<0.0001) | (0.001) | (ns) | | | |
| DNAmCD8Tcell | 0.014 | 0.013 | −0.002 | ns | — | — |
| | (0.019) | (0.069) | (12%) | | | |
| DNAmCD4Tcell | 0.185 | 0.152 | −0.033 | ns | — | — |
| | (0.185) | (0.069) | (18%) | | | |
| DNAmNKcell | 0.063 | 0.063 | −0.0003 | ns | — | — |
| | (0.039) | (0.054) | (0.5%) | | | |
| DNAmGranulocyte | 0.667 | 0.653 | −0.015 | ns | — | — |
| | (0.131) | (0.103) | (2.2%) | | | |

¹Mean (SD);
²R\hat{ }2 (t distribution)

Conclusion: NE3107 treatment for 3 months significantly decreased biological age compared to chronological age, and epigenetic measures correlating with metabolic parameters known to be associated with cognitive decline. DNAm decreased in monocytes from subjects treated with NE3107, suggesting that monocyte lineage (including microglia) DNAm may be the keystone in diseases related to metabolic inflammation, including MCI and AD. The data suggests that NE3107 may decrease methylation in promoters of monocyte/macrophage regulators that can decrease inflammation and restore metabolic homeostasis in subjects with dementia.

### Example 2. Inflammatory Pathways of Neurodegenerative Mechanisms Linked to PD Progression

The great majority of Parkinson's disease (PD) is characterized as idiopathic and a minority have a known genetic basis most frequently linked to mutations in the neuronal protein, alpha-synuclein (SNCA) and genes associated with mitochondrial homeostasis. Over-production, misfolding, and aggregation of SNCA is a major contributor to neuronal oxidative stress and energy dyshomeostasis. Misfolded SNCA acts as a prion and is believed to have a role in disease spreading within the central nervous system (CNS), and there is evidence of transmission from the gut to the CNS as a possible means of disease initiation. A widely held view is that motor symptoms of the disease result primarily from low levels of the neurotransmitter, dopamine, secondary to the loss of dopaminergic neurons in the substantia nigra. This dopamine-centric view tends to understate the critical contribution of inflammation to disease expression. Although a threshold of 50%-80% reduction in dopamine levels is generally assumed necessary to cause motoric symptoms parkinsonian behavior can be observed with considerably less neurodegeneration in animal models of PD initiated with neuroinflammatory agents. Moreover, reduction of neuroinflammation and oxidative stress without dopaminergic therapy can improve mobility and decrease clinical signs of disease in animal models and humans. Important to the concept of anti-inflammatory therapy of PD is pharmaceutical acceptability, which extends to drug candidate safety for chronic use, blood-brain barrier permeability, and a mechanism of action that targets critical aspects of the inflammatory processes driving disease expression and progression.

Inflammation and oxidative stress are mutually inductive and drive pathophysiology in neurodegenerative diseases. In PD, misfolded over-expressed aggregated SNCA interacts with molecular pattern receptors, toll-like receptor 4 (TLR4), and the receptor for advanced glycation end products (RAGE), to activate inflammatory signaling cascades controlled by specific extracellular signal-regulated kinase-nuclear factor-kappa B (ERK-NFKB)-containing scaffolds that mediate tumor necrosis factor (TNF), interleukin 1b (IL-1b), interleukin-6 (IL-6), and other inflammatory cytokine production. These inflammatory signaling mechanisms are independent from NFKB-ERK homeostatic signaling pathways that control cell proliferation, long-term potentiation, and insulin signaling (such as Ras/Raf/MEK/ERK).

Activation of inflammatory pathways triggers inducible nitric oxide synthase (iNOS) synthesis that promotes formation of reactive nitrogen and oxygen species that impact mitochondrial cytochrome efficiency to decrease energy production, increase calcium currents, and generate more reactive oxygen species. These oxidation species activate NFKB and calmodulin kinase in a cycle that tends to feed forward, creating a state of chronic inflammation and oxidative stress. Mitochondrial dysfunction in various forms is a primary driver of PD pathophysiology. Activated microglia and astrocytes play a prominent role in PD pathology and progression through maintenance of an inflammatory milieu. Accumulating evidence demonstrates the reactive oxygen species (ROS) and pro-inflammatory cytokines produced by microglia are engaged in the induction and perpetuation of the neurodegenerative processes in PD.

High energy demands of dopaminergic neurons make them especially vulnerable to deleterious effects of mitochondrial dysfunction. Maintenance of protein homeostasis is an energy intensive process. Endoplasmic reticulum (ER) stress results when cells lack sufficient reducing power to properly guide newly synthesized protein folding, preserve function and prevent aggregation. Under conditions of ER stress, SNCA can be misfolded and aggregated into oligomeric sheets that are toxic to mitochondria in addition to activating molecular pattern receptors and inflammatory pathways.

Insulin signaling plays a vital role in neuronal energy homeostasis and neuron survival. Inflammatory activation of mitogen associated protein kinases (MAPK) can inhibit insulin signaling (induce insulin resistance) through phosphorylation of various serine residues on insulin receptor substrate 1 and 2 (IRS-1/2) that interfere with insulin receptor tyrosine phosphorylation or interactions of IRS-1/2 with the insulin receptor or other proteins in the signaling complex. Inflammation triggered by SNCA can thus contribute to insulin resistance that hinders mitochondrial function and promotes oxidative and ER stress and feeds forward to increased SNCA dyshomeostasis. Interestingly, intranasal insulin has promotoric activity in PD, and insulin resistance has been linked to PD cognitive symptoms as well. It is reported that 60%-80% of PD subjects have insulin resistance. Several anti-diabetic agents are in clinical evaluation for potential benefits to PD patients (clinicaltrials.gov; NCT04251585, NCT02953665, NCT04232969).

Inflammatory chemokines promote infiltrating lymphocytes and macrophages that contribute to the inflammatory milieu in PD. An adaptive immune response of T cells recognizing SNCA peptides contributes to the neurodegenerative process with reactive astrocytes potentially acting as antigen-presenting cells that may also facilitate the spread of SNCA aggregates. Therefore, infiltrating lymphocytes have the potential to contribute to PD pathophysiology through multiple mechanisms, and decreasing inflammatory cell infiltration may have a significant impact on disease.

ERK mediated neuroinflammation is causally associated with levodopa-induced dyskinesia (LID), as described elsewhere herein, and genetic and pharmacological modulation of ERK activation reduces LID in rodent models. Furthermore, inflammatory signals mediated by inflammatory NFKB-ERK signaling are linked to excitotoxicity and well-established neurodegenerative mechanisms linked to PD progression. This pathogenic effector mechanism in PD patients is recapitulated in a valid disease model, elicited by injection of 1-methyl-4-phenyl-1,2,3,6-tetrahydropyridine (MPTP) into marmoset monkeys (Callithrix jacchus). MPTP is a blood-brain permeable substrate for the dopamine transporter that is metabolized to the mitochondrial toxin, 1-methyl-4-phenylpyridinium (MPP⁺) by monoamine oxidase in astocytes and selectively taken up by neurons through monoamine transporters. MPTP administration to animals (and humans) produces Parkinson's-like symptoms that result from loss of dopaminergic cells (neurodegeneration) in the substantia nigra pars compacta (SNpc) and the consequent reduced striatal dopamine concentrations and neuroinflammation. Selective injury of dopaminergic (DA) cells after MPTP intoxication is immediately followed by the clustering of microglia around injured neurons. The reaction of microglia expressed myeloperoxidase (MPO) and H₂O₂ with structurally-related ortho-methoxy-substituted catechols, such as apocynin and vanillic acid, generates reactive intermediates that bind to free thiol groups. MPO is up regulated in activated brain microglia cells of PD patients and in the MPTP-induced animal model. As with Parkinson's disease (which is unique to humans) dopamine administration to MPTP-treated animals can significantly improve motor control, but does not decrease inflammatory mechanisms, and thus does not modify disease processes or slow progression in animal models. Therefore, selective inhibitors of microglia ROS production or modulation of ERK activity will be potentially effective medicines for PD aiming at mitigation or prevention of DA neuron degeneration and reduction of LID. Experiments with apocynin have indeed shown that this principle can be used to prevent the assembly and activation of the ROS generating NADPH oxidase in the MPTP marmoset model.

### Example 3: Effect of Potential Drug Candidate NE3107 on TNFa-Stimulated TNFR1 ERK-Dependent Phosphorylation

THP-1 human monocytes were grown in complete growth medium (ATCC-formulated RPMI-1640 Medium, Catalog No. 30-2001), 2-mercaptoethanol to a final concentration of 0.05 mM and fetal bovine serum to final concentration of 10%. THP-1 human monocytes were then seeded into 10 mL complete growth medium at 0.4×10⁶/mL in a T25 flask and were grown to 0.8×10⁶/mL in about 1 to 2 days. The cells were then starved in 0.5% FBS RPMI for 16 h. The THP-1 monocytes were then treated with 100 nM 17α-ethynylandrost-5-ene-3ϕ3,7ϕ3,17ϕ3-triol (NE3107), 1 μM IKK-16 (IKK inhibitor), 1 μM Tpl2 Kinase inhibitor (TPL2i) or 0.1% DMSO (vehicle). Cells were stimulated with stimulated with 50 ng/mL TNFa and cell lysates were collected at 0, 2, 5, 10, 15, and 30 minutes after TNFa stimulation. Total cell lysates were subjected to Western blotting as described in Lu et al., Mol. Endocrinol. 22 (2008), 760-771. Relative band intensities were quantified by densitometry scanning. The results are given as fold change with respect to untreated cells in Table 2.

TABLE 2

| | Fold vs DMSO | Fold vs DMSO |
|---|---|---|
| | control | control |
| | P-TNFR1/TNFR1 | P-TNFR1/Actin |
| | in Gel2 | in Gel2 |
| DMSO 0 min | 1.00 | 1.00 |
| TNFa 0 min | 1.54 | 1.89 |
| IKKi + TNFa 0 min | 2.92 | 2.09 |
| TPL2i + TNFa 0 min | 0.69 | 0.71 |
| NE3107 + TNFa 0 min | 0.27 | 0.32 |
| TNFa 15 min | 2.93 | 3.53 |
| IKKi + TNFa 15 min | 0.59 | 0.92 |
| TPL2i + TNFa 15 min | 0.37 | 0.40 |
| NE3107 + TNFa 15 min | 0.57 | 0.79 |
| TNFa 30 min | 2.86 | 1.84 |
| IKKi + TNFa 30 min | 0.30 | 0.49 |
| TPL2i + TNFa 30 min | 0.08 | 0.13 |
| NE3107 + TNFa 30 min | 0.17 | 0.23 |

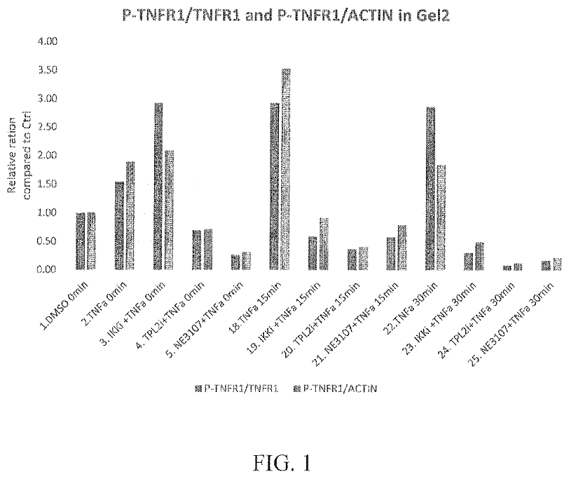

FIG. 1 demonstrates that cells stimulated by TNF show increased gene expression compared to the control. Treatment with NE3107 reduced gene expression compared to the control.

### Example 4

In this example, the clinical outcomes from a Phase 3, randomized, placebo-controlled trial of NE3107 (17α-ethynylandrost-5-ene-3ϕ3,7ϕ3,17ϕ3-triol) in subjects with mild to moderate probable Alzheimer's disease.

### BACKGROUND

Recently, the roles of inflammation and insulin resistance in neurodegeneration have become better appreciated. NE3107, an oral small molecule, blood-brain permeable anti-inflammatory insulin sensitizer that binds extracellular signal-regulated kinase, has been shown to selectively inhibit inflammation-driven ERK- and NF-kB-stimulated inflammatory mediators, including TNF-α, without inhibiting their homeostatic functions. We describe the rationale and design of NM101, the first randomized, multicenter Phase III clinical study to examine the safety and efficacy of 30-week treatment with NE3107 versus placebo in elderly adults with mild-to-moderate Alzheimer's disease. Patients (316) will be randomized in a 1:1 ratio. The co-primary end points measure cognitive function and functional and behavioral characteristics (Clinical Dementia Rating Scale Sum of Boxes, CDR-SB) and cognitive function (ADAS Cog12). Trial registration number: NCT04669028 (Clinicaltrials.gov).

### Trial Design

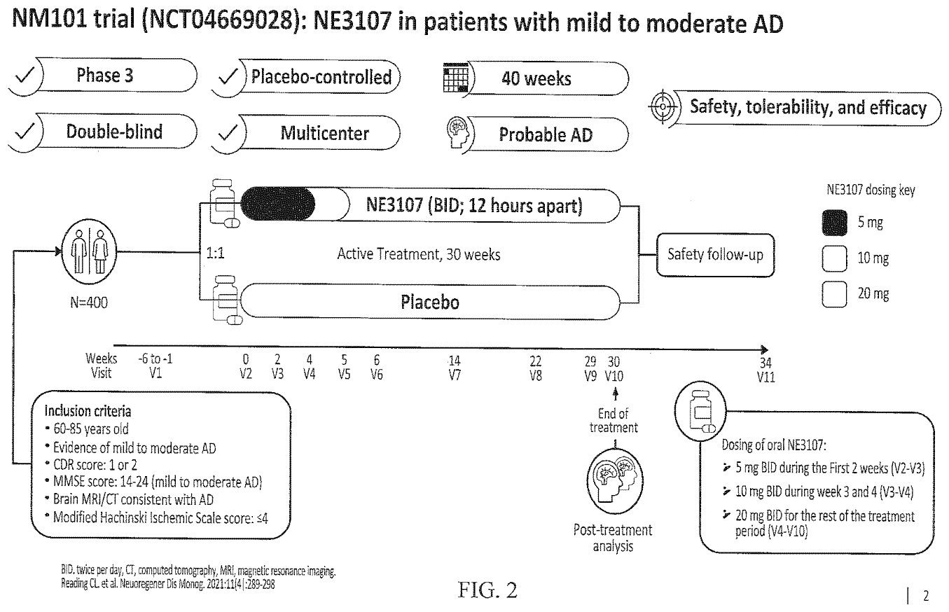

The trial design is illustrated in FIG. 2.

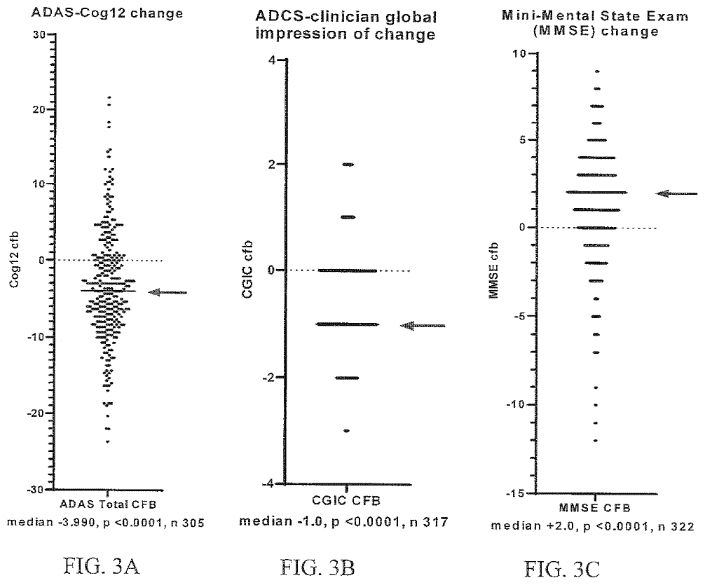

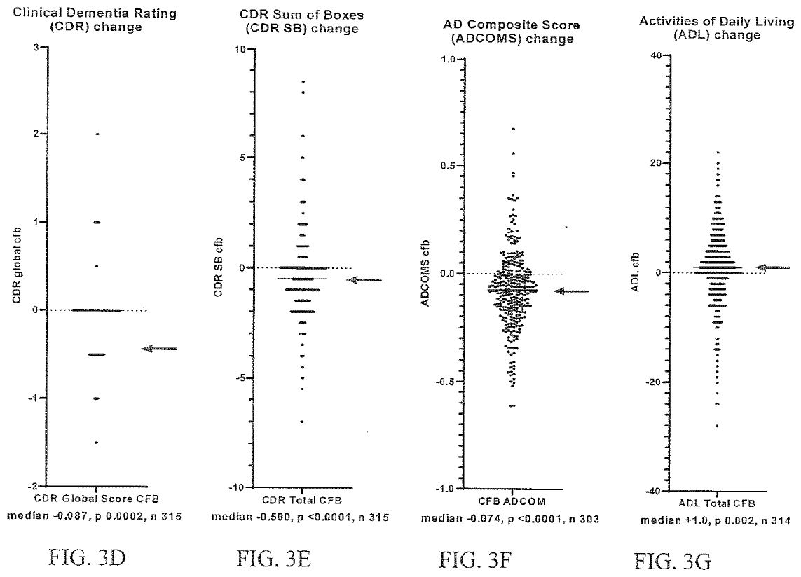

During the Phase 3 trials, the median improvement in various blinded assessments were measured. The results are described in Table 3 and FIGS. 3A-3G.

TABLE 3

| Spearman r* | CGIC | MMSE | CDR | CDR SB | ADCOMS | ADL |
|---|---|---|---|---|---|---|
| Cog12 | +0.24 | +0.46 | +0.23 | +0.23 | −0.24 | −0.40 |
| CGIC | | −0.50 | | | +0.46 | −0.40 |
| MMSE | | | −0.38 | −0.42 | −0.63 | +0.34 |
| CDR SB | | | | | +0.79 | −0.21 |
| ADCOMS | | | | | | −0.23 |

Similar distributions were observed for APOE4+/−, mild/moderate Alzheimer's disease, male/female, and older/younger participants.

Subjects metabolic and correlation changes were analyzed. Many of the genes associated with LOAD (late onset Alzheimer's disease) are related to cholesterol metabolism, which is decreased in AD neuron membranes. Insulin and glycemic controls are known to be involved in neurodegeneration. Increased insulin and HOMA2-% B cell function, and decreased HOMA2% insulin sensitivity resulted in no cases of hypoglycemia. Increased mean amplitude of glycemic excursion (MAGE from CGM) increased risk of Alzheimer's disease progression. Leptin is an anti-inflammatory, neuroprotective and is decreased in AD. Leptin DNAm was observed to increase. The results are described in Tables 4 and 5.

TABLE 4

| | | | HOMA2-% | HOMA2-% | | |
|---|---|---|---|---|---|---|
| | Cholesterol | Insulin | B | S | MAGE | Leptin |
| Change | +3.0 mg/dL | +1.9 | +13.5%, * | −21.2%, * | −0.57 | |
| from | | μIU/mL, * | | | mg/dL | |
| Baseline | | | | | | |
| V10 | | | | | | 29.2 |
| median | | | | | | ng/mL |

* P < 0.0001

TABLE 5

| Spearman r | CGIC | ADL | Cog12 | MMSE |
|---|---|---|---|---|
| Cholesterol | +0.170** | +0.130* | −0.164** | |
| MAGE | +0.253* | | | |
| Leptin# | | | −0.264* | 0.344* |

*p < 0.05,
**p < 0.005,
***p < 0.0001,
#excludes obese subjects

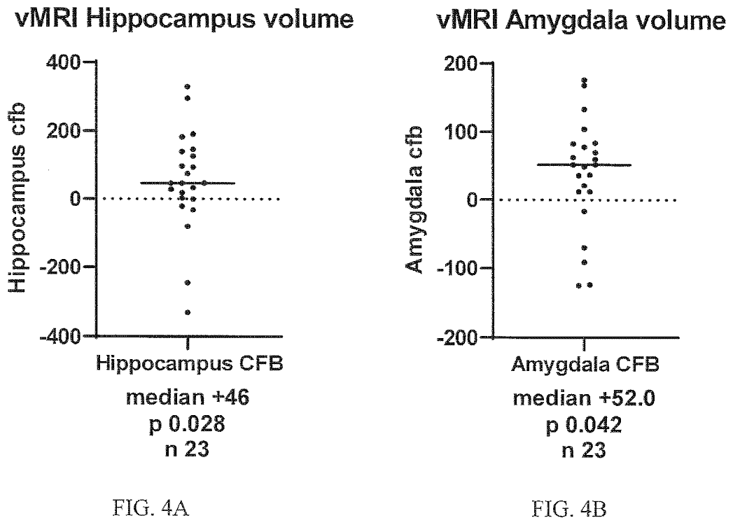

Subjects were analyzed using imaging sub-studies vMRI on 23 subjects. It was observed that the volume was increased in the hippocampus and amygdala. In addition, it was observed that a decreased CGIC was correlated with decreased hippocampi volume and increased MCP-1 was correlated with increased whole cortex. The results are described in FIGS. 4A-4B and Table 6.

TABLE 6

| Spearman r | Whole Cortex | Hippocampus |
|---|---|---|
| CGIC | −0.458** | |
| MCP-1 | | +0.713** |

**p < 0.05

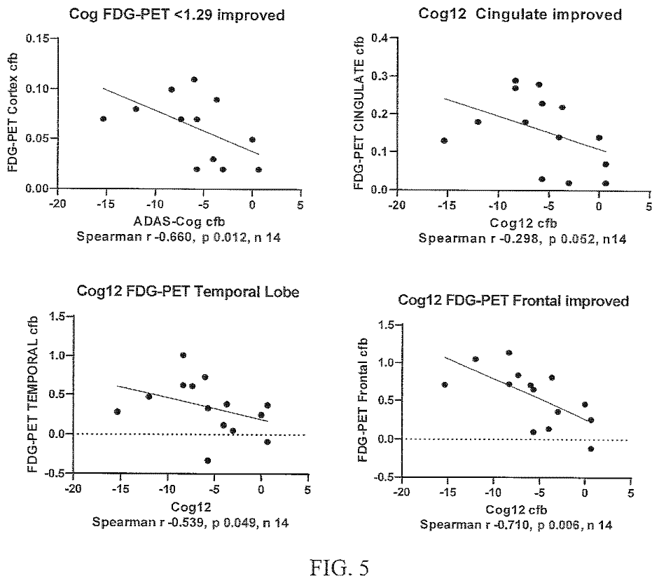

Subjects were analyzed using a fluorodeoxyglucose (FDG)-positron emission tomography (PET) imaging scans. FDG-PET standardized uptake value ratios (“suvr”) were increased in 14/24 subjects with a baseline whole cortex suvr <1.29. This data correlates with Cog12 and ADL with suvr improvements. Cholesterol increases trended with increased Cingulate suvr. The results are described in Table 7 and FIG. 5.

TABLE 7

| Spearman r | Cortex | Cingulate | Temporal | Parietal | Frontal |
|---|---|---|---|---|---|
| PET (+) | −0.660** | −0.534* | −0.540 | −0.197 | −0.710*** |
| Cog12 | | | | | |
| PET (−) | −0.298 | −0.298 | −0.115 | −0.165 | −0.114 |
| Cog12 | | | | | |
| PET (+) | +0.546** | +0.623** | +0.523** | −0.197 | +0.358 |
| ADL | | | | | |
| PET (−) | −0.146 | −0.298 | −0.115 | −0.164 | −0.114 |
| ADL | | | | | |
| Cholesterol | | +0.424* | | | |

*p < 0.1,
**p < 0.05,
***p < 0.01

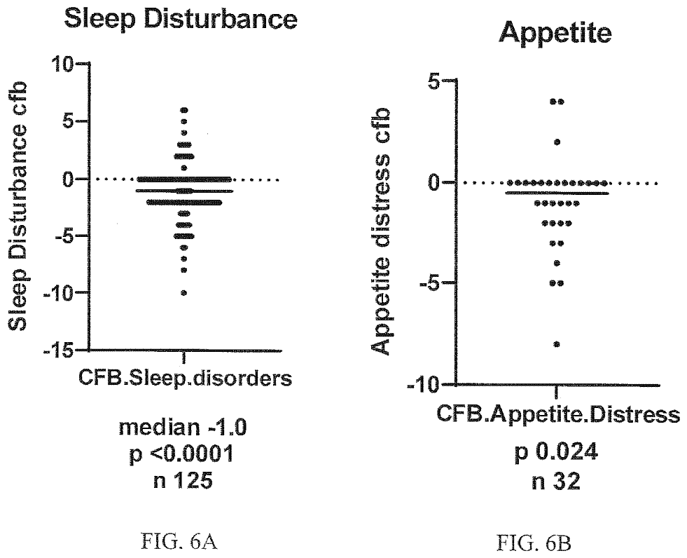

Subjects were analyzed for neuropsychiatric inventory. It was observed that overall improvement was seen in sleep (−1.0, p<0.0001) and appetite (−1.0, p<0.023). An improvement in appetite was also correlated with decreased Cog12, CDR SB and ADCOMS, with increased Alzheimer's disease. A decrease in anxiety was correlated with an increase ADL and decreased CGIC. Sleep improvement was correlated with a decrease in CGIC and decreased in TNFa. The results are described in Table 8 and FIGS. 6A-6B.

TABLE 8

| Spearman r | Cog12 | CG12 | CDR SB | ADCOMS | ADL | TNFa |
|---|---|---|---|---|---|---|
| Appetite | +0.54*** | | +0.36** | +0.36* | −0.65**** | |
| Anxiety | | +0.15* | | | −0.14* | |
| Sleep | | +0.22** | | | | +0.24* |

*p < 0.10,
**p < 0.05,
***p < 0.01,
****p < 0.001

The study overall had a very low rate of adverse effects (AEs) reported and only 8 subjects discontinued due to a reported AE (1.8%). Of the 439 subjects enrolled in the study, 156 experienced 1 or more AEs (35.5%). There were only 43 (9.8%) related AEs with the majority 42 (0.6%) categorized as non-serious AEs by the principal investigator. The study also had very low rate of serious AEs reported. There were 12 (2.7%) reported for the duration of the study. Only 1 of the serious AEs reported resulted in death. As this is a blinded analysis it is not known if the subjects was on IP or placebo.

While certain embodiments of the inventions have been described, such embodiments have been presented by way of example, and are not intended to limit the scope of the disclosure. Indeed, the compounds, compositions, processes, and methods described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed. For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein. The scope of the present disclosure is not intended to be limited by the specific disclosures of preferred embodiments.

What is claimed is:

1. An in vitro screening method to identify a potential drug candidate capable of treating, preventing, reducing, or ameliorating a disorder or disease, comprising:
(i) providing a sample for stimulation selected from the group consisting of a cell, tissue, blood, monocytes, microglia, macrophages, adipocytes, neuroblastoma, pheochromocytoma, and Lund human mesencephalic (LUHMES) cells;
(ii) stimulating the sample with an agonist to induce a phenotype or phenotypic reaction, wherein the phenotype or phenotypic reaction substantially corresponds to a disease or condition associated with a biological clock;
(iii) contacting the sample exhibiting the phenotype or phenotypic reaction with one or more potential drug candidates;
(iv) determining a responsive change in the phenotype or phenotypic reaction of the sample; and
(v) providing the one or more potential drug candidates to a subject to treat, reduce, prevent, or ameliorate a disease or condition associated with a biological clock in a subject.

2. The method of claim 1, wherein the responsive change is a decrease or loss in the phenotype and the decrease or loss is indicative that the potential drug candidate is capable of preventing, reducing, or ameliorating a neurodegenerative disorder or disease.

3. The method of claim 2, wherein the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism.

4. The method of claim 2 or 3, wherein the neurodegenerative disorder or disease is Parkinson's disease.

5. The method of claim 2 or 3, wherein the neurodegenerative disorder is Alzheimer's disease.

6. The method of any one of claims 1 to 5, wherein the disease or condition associated with biological clocks in the subject in need thereof is based on modulation of DNA methylation of genes associated with biological clocks.

7. The method of claim 6, wherein the disease or condition associated with the biological clock in the subject in need thereof is associated with genes or genomic regions hypermethylated with age.

8. The method of claim 6, wherein the disease or condition associated with a biological clock in the subject in need thereof is associated with genes or genomic regions hypomethylated with age.

9. The method of claim 6, wherein the disease or condition associated with inflammatory TNF signaling.

10. The method of claim 6, wherein the disease or condition associated with inflammatory NF-kB signaling.

11. The method of any one of claims 1 to 8, wherein the disease or condition associated with a biological clock in the subject in need thereof is associated with Tau phosphorylation.

12. The method of any one of claims 1 to 8, wherein the disease or condition associated with a biological clock in the subject in need thereof is associated with hyperglycemia.

13. The method of any one of claims 1 to 8, wherein the disease or condition associated with the biological clock in the subject in need thereof is associated with hyperinsulinemia.

14. The method of any one of claims 1 to 8, wherein the disease or condition associated with the biological clock in the subject in need thereof is associated with obesity.

15. The method of any one of claims 1 to 8, wherein the disease or condition associated with the biological clock in the subject is connected to leptin.

16. The method of claim 15, wherein the subject experiences a decrease between about a 5% to about a 100% reduction in conditions or symptoms connected to leptin after administration of the one or more drug candidate and at least one pharmaceutically acceptable excipient.

17. The method of any one of claims 1 to 8, wherein the disease or condition associated with a biological clock is associated with a subject's SkinBloodAge.

18. The method of claim 17, wherein the subject experiences a decrease between about a 5% to about a 100% reduction in conditions or symptoms connected to SkinBloodAge after administration of the one or more drug candidate and at least one pharmaceutically acceptable excipient.

19. The method of any one of claims 1 to 8, wherein the disease or condition associated with the biological clock is associated with a subject's dinucleotide (CpG) methylation in association with leptin promotor (DNAmLeptin).

20. The method of claim 19, wherein the subject experiences a decrease between about a 5% to about a 100% reduction in conditions or symptoms connected to DNAmLeptin after administration of the drug candidate and at least one pharmaceutically acceptable excipient.

21. The method of any one of claims 1 to 8, wherein the disease or condition associated with a biological clock is associated with a subject's DNAmPack Years.

22. The method of claim 21, wherein the subject experiences a decrease between about a 5% to about a 100% reduction in conditions or symptoms connected to DNAmPackYears after administration of the drug candidate and at least one pharmaceutically acceptable excipient.

23. A method to treat, prevent, reduce, or ameliorate a neurodegenerative disorder or disease, comprising:
(i) providing a sample for stimulation selected from the group consisting of a cell, tissue, blood, monocytes, microglia, macrophages, adipocytes, neuroblastoma, pheochromocytoma, and Lund human mesencephalic (LUHMES) cells;
(ii) stimulating the sample with an agonist to induce a phenotype or phenotypic reaction, wherein the phenotype or phenotypic reaction substantially corresponds to a disease or condition associated with a biological clock;
(iii) contacting the sample exhibiting the phenotype or phenotypic reaction with one or more potential drug candidates;
(iv) determining a responsive change in the phenotype or phenotypic reaction of the sample; and
(v) providing the one or more potential drug candidates to a subject to treat, reduce, prevent, or ameliorate a disease or condition associated with a biological clock in a subject.

24. The method of claim 23, wherein the responsive change is a decrease or loss in the phenotype and the decrease or loss is indicative that the potential drug candidate is capable of preventing, reducing, or ameliorating a neurodegenerative disorder or disease.

25. The method of claim 24, wherein the neurodegenerative disorder or disease is selected from the group consisting of Alzheimer's disease, Parkinson's disease, levodopa-induced dyskinesia (LID), amyotrophic lateral sclerosis (ALS), frontotemporal dementia (FTD), hippocampal sclerosis of aging (HS-Aging), chronic traumatic encephalopathy (CTE), progressive supranuclear palsy, multiple system atrophy, corticobasal degeneration and vascular parkinsonism.

26. The method of claim 23 or 24, wherein the neurodegenerative disorder or disease is Parkinson's disease.

27. The method of claim 23 or 24, wherein the neurodegenerative disorder is Alzheimer's disease.

28. The method of any one of claims 23 to 27, wherein the disease or condition associated with biological clocks in the subject in need thereof is based on modulation of DNA methylation of genes associated with biological clocks.

29. The method of claim 28, wherein the disease or condition associated with the biological clock in the subject in need thereof is associated with genes or genomic regions hypermethylated with age.

30. The method of claim 28, wherein the disease or condition associated with the biological clock in the subject in need thereof is associated with genes or genomic regions hypomethylated with age.

31. The method of claim 28, wherein the disease or condition associated with inflammatory TNF signaling.

32. The method of claim 28, wherein the disease or condition associated with inflammatory NF-kB signaling.

33. The method of any one of claims 23 to 30, wherein the disease or condition associated with the biological clock in a subject in need thereof is associated with Tau phosphorylation.

34. The method of any one of claims 23 to 30, wherein the disease or condition associated with the biological clock in a subject in need thereof is associated with hyperglycemia.

35. The method of any one of claims 23 to 30, wherein the disease or condition associated with the biological clock in a subject in need thereof is associated with hyperinsulinemia.

36. The method of any one of claims 23 to 30, wherein the disease or condition associated with the biological clock in a subject in need thereof is associated with obesity.

37. The method of any one of claims 23 to 30, wherein the disease or condition associated with the biological clock in a subject is connected to leptin.

38. The method of claim 37, wherein the subject experiences a decrease between about a 5% to about a 100% reduction in conditions or symptoms connected to leptin after administration of the drug candidate and at least one pharmaceutically acceptable excipient.

39. The method of any one of claims 23 to 30, wherein the disease or condition associated with the biological clock is associated with a subject's SkinBloodAge.

40. The method of claim 39, wherein the subject experiences a decrease between about a 5% to about a 100% reduction in conditions or symptoms connected to SkinBloodAge after administration of the drug candidate and at least one pharmaceutically acceptable excipient.

41. The method of any one of claims 23 to 30, wherein the disease or condition associated with a biological clock is associated with a subject's dinucleotide (CpG) methylation in association with leptin promotor (DNAmLeptin).

42. The method of claim 41, wherein the subject experiences a decrease between about a 5% to about a 100% reduction in conditions or symptoms connected to DNAmLeptin after administration of the drug candidate and at least one pharmaceutically acceptable excipient.

43. The method of any one of claims 23 to 30, wherein the disease or condition associated with a biological clock is associated with a subject's DNAmPackYears.

44. The method of claim 43, wherein the subject experiences a decrease between about a 5% to about a 100% reduction in conditions or symptoms connected to DNAmPack Years after administration of the drug candidate and at least one pharmaceutically acceptable excipient.

45. The method of any one of claims 1 to 44, wherein the sample for stimulation further includes cells selected from induced pluripotent stem cells, mesenchymal stem cells, or endothelial cells.

46. The method of any one of claims 1 to 45, wherein the agonist used for stimulation is selected from a group consisting of cytokines, chemokines, growth factors, or environmental stressors like oxidative stress or hypoxic conditions.

47. The method of any one of claims 1 to 46, wherein the potential drug candidate is selected from the group consisting of small molecules, peptides, antibodies, RNA-based therapies, or gene-editing tools.

48. The method of any one of claims 1 to 47, wherein the determining of a responsive change in phenotype or phenotypic reaction includes measuring changes in cellular metabolism, morphology, or signaling pathways.

49. The method of claim 48, wherein the change in cellular signaling pathways includes modulation of pathways associated with autophagy, apoptosis, or senescence.

50. The method of any one of claims 1 to 49, wherein the disease or condition associated with the biological clock includes conditions related to cardiovascular health, such as atherosclerosis or hypertension.
